(19) United States
(12) Patent Application Publication
(10) Pub. No.: US 2013/0203166 A1
(43) Pub. Date: Aug. 8, 2013

(54) STIMULATION OF MULTIPOTENCY OF MESENCHYMAL STEM CELLS BY CHEMOKINE CCL5

(71) Applicant: PROTOBIOS LLC, Tallinn (EE)

(72) Inventors: Mari-Liis Kauts, Tallinn (EE); Alla Piirsoo, Tallinn (EE)

(73) Assignee: PROTOBIOS LLC, Tallinn (EE)

(21) Appl. No.: 13/761,069

(22) Filed: Feb. 6, 2013

Related U.S. Application Data
(60) Provisional application No. 61/595,278, filed on Feb. 6, 2012.

(51) Int. Cl. C12N 5/071

(52) U.S. Cl. CPC C12N 5/0662
USPC 435/366

(57) ABSTRACT

Methods of stimulating multipotency, proliferation and differentiation of isolated mesenchymal stem cells (MSCs), which permit more effective differentiation and integration of such cells into host tissues. The method includes providing an in vitro cell population of MSCs and administering CCL5 chemokine. Preferably CCL is administered in an amount sufficient to induce expression of one or more multipotency related genes selected from the group consisting of OCT3/4, NANOG, SOX 2, KLF4, and SOX9.

### CROSS-REFERENCE TO RELATED APPLICATIONS

This application claims benefit of priority to U.S. provisional patent application Ser. No. 61/595,278 filed Feb. 6, 2012; the content of which is herein incorporated by reference in its entirety.

### TECHNICAL FIELD

The invention relates to the use of chemokine CCL5 (RANTES) to stimulate mesenchymal stem cells following isolation from the adipose tissue and more specifically to methods of inducing expression of NFkB-dependent genes or multipotency related genes in mesenchymal stem cell populations, stimulation of proliferation and differentiation through the administration of CCL5.

### BACKGROUND OF THE INVENTION

Adult mesenchymal stem-like cells (MSC) are referred as postnatally isolated naive or ex vivo expanded plastic-adherent cells with high proliferative capacity able to differentiate in vitro and preferably also in vivo into different mesodermal lineages, such as adipocytes, chondrocytes, osteoblasts. Those features, enhanced by MSC nonimmunogenic phenotype and their ability to migrate to injured or inflamed sites create a strong platform for their clinical applications, such as tissue regeneration, treatment of immune, cardiovascular and degenerative diseases (osteoarthritis and osteoporosis) (Gimble et al., 2007; Parekkadan and Milwid, 2010).

Cells with MSC-like properties have been found in almost all tissues and organs, e.g. peripheral blood, bone marrow, umbilical cord, adipose tissue, muscle, liver, thymus, dental pulp, hair follicles and skin (Gimble et al., 2007; Parekkadan and Milwid, 2010). Nowadays, white adipose tissue is considered as one of the most promising sources of adult MSC because of its abundance and relatively simple harvesting. Growth kinetics, immunogenic characteristics, differentiation potential, senescence ratio and in vivo angiogenic activity of adipose tissue derived mesenchymal stem-like cells (AdMSCs) are comparable with MSCs obtained from other sources; however, several differences have been reported (De Ugarte et al., 2003; Noel et al., 2008).

Adipose tissue contains mature adipocytes, fibroblasts, pericytes, smooth muscle cells, macrophages, lymphocytes, regulatory T cells, preadipocytes and MSC (Gimble et al., 2007; Madonna et al., 2009). Widely used methods of AT-MSC isolation include separation of AT stromal vascular fraction (SVF), its positive or negative sorting according to the suggested by the International Society for Cellular Therapy immunophenotype, and selection of a plastic adherent population (Dominici et al., 2006). Nowadays, it is clear that this immunophenotype fits other cell types too (Tallone et al., 2011). Despite the fact that the SVF is a heterogeneous cell population, selection of a plastic adherent population and subsequent short-term expansion enrich the cells expressing a stromal immunophenotype, comparing with the heterogeneity of the crude SVF (Dominici et al., 2006; Tallone et al., 2011).

It has been suggested that sternness of embryonic stem cells is supported by the regulatory networks containing in their core the key transcription factors (OCT4, NANOG, SOX2 and KLF4) that are associated with pluripotency and form the basis for regulation of stem cell renewal and differentiation (Ng and Surani, 2011); (Meshorer and Misteli, 2006; Pan and Thomson, 2007). Molecular analysis of MSCs has identified similar regulatory networks. Stemness maintenance of MSCs is related to several transcription factors such as KLF4 (Saulnier et al., 2011), OCT4 (Greco et al., 2007) and SOX2 (Park et al., 2011). Also, other transcription factors known to be responsible for embryonic stem cell maintenance are expressed in MSCs (Riekstina et al., 2009).

One of the potential clinical applications of isolated and cultured MSC relies on their capacity for targeted and controlled migration and engraftment to sites of injury, inflammation and tumours (Chamberlain et al., 2007). Furthermore, it is believed that under inflammatory conditions endogenous MSC are activated and induced for directional movement to sites of injury where they participate in tissue regeneration processes. Signals of inflammation are in part mediated by chemokines. Chemokines are secreted signalling molecules regulating numerous functions of cells such as proliferation, establishment of cellular polarity, migration, angiogenic capacity and differentiation. Besides, a number of experiments have revealed that chemokines may elicit both anti- and pro-apoptotic signals in a concentration and cell-type dependent manner (Khan et al., 2008; Kochetkova et al., 2009; Liu et al., 2007; Sanchez-Sanchez et al., 2004; Vlahakis et al., 2002). Chemokines stimulate the target cells through binding to their receptors belonging to G-protein-coupled receptor family.

CCL5 or RANTES is one of the most studied inflammatory chemokine. CCL5 binds with similar affinity to three receptors—CCR1, CCR3 and CCR5. CCR1, CCR3 and CCR5 genes form a gene cluster on chromosome 3p suggesting that they may be expressed in the same cells. The multiple roles of CCL5 and its receptors are better understood in the functioning of the immune system. CCL5 activates T lymphocytes, monocytes and macrofages inducing their migration to the sites of inflammation. Besides, CCL5 may stimulate proliferation of lymphocytes and positively regulate T-cell differentiation (Luther and Cyster, 2001). Additionally, a number of studies have revealed association of CCL5 and its receptors with several types of malignant cancer, including myeloma, breast cancer, and melanoma (Zlotnik, 2006). Therefore CCL5 receptors are considered as potential therapeutic targets for the limitation of specific malignant diseases.

It has been shown that bone marrow derived MSCs express at least one functional CCL5 receptor (CCR1) (Fox et al., 2007; Honczarenko et al., 2006). Stimulation of the cells by CCL5 leads to activation of small GTPases such as RhoA, Rac1 and Cdc42 and increase of cyclin D1 and c-Myc production that suggests possible roles of those chemokines/chemokine receptor systems in regulation of cells survival, proliferation and migration. Receptors for CCL5 and molecular mechanisms of CCL5 signalling in AdMSCs have not been described yet. It is not yet clear whether CCL5/CCR1/CCR3/CCR5 axes participate in regulation of survival and proliferation of AdMSCs. Also, it has not been investigated yet, whether the expression of chemokine receptors is attributable to the stem cells that should also express the multipotency genes OCT4, SOX2, NANOG and KLF4.

### BRIEF SUMMARY OF THE INVENTION

We have developed a protocol to stimulate multipotency of mesenchymal stem cells that creates an opportunity to differentiate MSCs more efficiently towards different cell lineages used for different cell therapy applications. Specifically, the methods provide the administration of chemokine CCL5, which interacts with its receptors CCR1, CCR3 and CCR5 thereby stimulating MSCs. Stimulation of MSCs with CCL5 leads to induction of NFkB signalling pathway in an ERK and AKT kinases-dependent manner. Also, CCL5 treatment stimulates expression of key transcription factors associated with multipotency such as OCT4, NANOG and SOX2. CCL5 induced pathways stimulate proliferation, and differentiation of MSCs.

In one aspect of the invention a method of increasing expression of a chemokine receptor in a population of mesenchymal stem cells is provided. The method includes providing an in vitro population of human mesenchymal stem cells; and administering CCL5 to the population in an amount sufficient to increase expression of a chemokine receptor selected from the group consisting of CCR1, CCR2 and CCR5 in the population. In the preferred embodiment, the method increases expression of CCR1, CCR3 and CCR5 in the population of cells. In some embodiments, the population of cells is cultured for about 48 hours after the step of administering CCL5 to achieve a higher level of expression of the chemokine receptor compared to a same population of cells cultured for about 24 hours and for about 72 hours after the step of administering CCL5.

In a related aspect, a method of increasing expression of a nuclear factor kappa-light chain enhancer of activated B cells (NFkB) dependent gene selected from the group consisting of interleukin-6 (IL-6), matrix metalloproteinase 1 (MMP1), and matrix metalloproteinase 9 (MMP9) in a population of mesenchymal stem cells is provided. The method includes providing an in vitro population of human mesenchymal stem cells; and administering CCL5 to the population in an amount sufficient to increase expression of a NFkB-dependent gene selected from the group consisting of IL-6, MMP1, and MMP9. In preferred embodiments expression of an anti-apoptotic gene SURVIVINE is also increased. In some embodiments the population of cells is cultured for about 8 hours after administering CCL5 to obtain a higher level of expression of IL-6, MMP1, MMP9 and SURVIVINE and for about 24 hours to obtain a higher level of expression of matrix metalloproteinase 2 (MMP2).

In another related aspect a method of inducing expression of multipotency related genes selected from the group consisting of octamer-binding transcription factor 3/4 (OCT3/4), NANOG, sex determining region Y box 2 (SOX 2), Krueppel-like factor 4 (KLF4), and sex determining region Y box 9 (SOX9) is provided. The method includes providing an in vitro population of human mesenchymal stem cells; and administering CCL5 to the population in an amount sufficient to induce expression of one or more multipotency related genes selected from the group consisting of OCT3/4, NANOG, SOX 2, KLF4, and SOX9. In some embodiments the population of cells is cultured for about 48 hours from the step of administering CCL5 to achieve a higher level of expression of OCT 3/4, NANOG, and SOX2 compared to about 8 hours, 24 hours and 72 hours after the step of administering CCL5.

In another related aspect of the invention a method of stimulating proliferation of mesenchymal stem cells is provided. The method includes providing an in vitro population of human mesenchymal stem cells; and administering CCL5 to the population in an amount sufficient to stimulate proliferation.

In another related aspect of the invention a method stimulating differentiation of mesenchymal stem cells is provided. The method includes providing an in vitro population of human mesenchymal stem cells; and administering CCL5 to the population in an amount sufficient to stimulate differentiation of mesenchymal stem cells. In some embodiments the population of cells is cultured for 12 hours with CCL5 followed by differentiation into adipogenic or chondrogenic lineages assessed accordingly by the expression levels of peroxisome proliferator-activated receptor gamma 2 (PPARγ2), adiponectin (ADIPOQ), complement factor D (CFD) and fatty acid binding protein 4 (FABP4) for adipogenic lineage and sex determining region Y box 9 (SOX9), collagen type II alpha1 (COL2A1), NK3 homobox 1 (NKX3.1) and matrix metalloproteinase 13 (MMP13) for chondrogenic lineage.

### BRIEF DESCRIPTION OF THE DRAWINGS

FIG. 1 is a photograph of an agarose gel depicting RT-PCR results using two independent pairs of primers for blood cells (BC) and adult mesenchymal stem cells (AdMSC).

FIG. 2 is graph depicting results from qRT-PCR normalized according to GAPDH mRNA expression level for CCR1, CCR2 and CCR3.

FIG. 3 is a graph depicting results from qRT-PCR normalized according to GAPDH mRNA expression level for IL-6, MMP1, MMP2, MMP9, SURVIVINE, BID, and BAXa.

FIG. 4 is a graph depicting results from qRT-PCR normalized according to GAPDH mRNA expression level for OCT 3/4, NANOG, SOX2, KLF4, and SOX9.

FIG. 5 is a graph depicting results from BrdU labeling experiment showing stimulatory effect of CCL5 on proliferation of AdMSCs

FIG. 6 is a graph depicting results from qRT-PCR normalized according to GAPDH mRNA expression level for PPARγ2, ADIPOQ, CFD and FABP4 as markers for adipogenesis.

FIG. 7 is a graph depicting results from qRT-PCR normalized according to GAPDH mRNA expression level for SOX9, COL2A1, NKX3.1 and MMP13 as markers for chondrogenesis.

### DETAILED DESCRIPTION OF PREFERRED EMBODIMENTS

The invention provides methods of stimulating multipotency of isolated mesenchymal stem cells (MSCs), which will permit more effective differentiation and integration of such cells into host tissues. In addition, the methods provide a basis for novel treatments using cells differentiated into cell types that do not develop using standard established procedures.

MSCs for use in the invention may be obtained from a variety of bodily fluids or tissues. Among these include peripheral blood, bone marrow, umbilical cord, muscle, liver thymus, dental pulp, hair follicles, skin or the like. In preferred embodiments, MSCs from adipose tissue are used due to its abundance and ease of harvest. MSCs may be used from a crude impure sample or may be isolated to provide a substantially pure sample containing about 90%, 95%, 99% or more MSCs. Methods for separating MSCs from surrounding adipose tissue may involve obtaining a stromal vascular fraction (SVF) followed by selection of a plastic adherent population followed by short term expansion to enrich cells expressing the stromal immunophenotype as demonstrated by Dominici et al. 2006 and Tallone et al. 2011.

Once a population of MSCs is obtained, the population is treated with CCL5 to induce multipotency. This is preferably achieved by maintaining the MSCs in culture in the presence of CCL5 for about 8 hours, more preferably about 12 hours, more preferably about 24 hours and most preferably about 48 hours, which as demonstrated in the examples and drawings results in increase expression of OCT3/4, NANOG and SOX2. SOX9 levels were found significantly elevated after 8 hours. Multipotency can be predicted using quantitative reverse transcription polymerase chain reaction (qRT-PCR) of multipotency related genes using suitable sense and anti-sense primers for each gene of interest using guidance provided throughout the examples.

As demonstrated in the examples, we show that AdMSCs secrete low levels of CCL5 detectable only during initial cultivation of cells until passage 4. AdMSCs express mRNA for CCL5 receptors CCR1, CCR2, CCR3 and CCR5. Expression of CCRs was also confirmed by FACS and IF analyses. AdMSCs are able to respond to CCL5 signal suggesting that CCL5 receptors are biologically functional. Stimulation of AdMSCs with CCL5 leads to induction of NFkB signalling pathway in an ERK and AKT kinases-dependent manner. As a result, expression of NFkB target genes such as IL-6, MMP1 and MMP9 is elevated. Besides, in response to CCL5, expression of anti-apoptotic gene SURVIVINE increases, whereas pro-apoptotic genes BID and BAXa are down-regulated. These data suggest that CCL5 may be a positive regulator of AdMSC proliferation inducing also anti-apoptotic effects. We show also that the level of expression of key transcription factors associated with multipotency (OCT4, NANOG and SOX2) correlates with the level of expression of CCL5 receptors in AdMSCs. Moreover, OCT4, SOX2 and NANOG expression levels as well as expression of CCR1, CCR3 and CCR5 increase in response to CCL5 stimulation. Taken together, our data suggest that multipotent cells possess functional receptors for CCL5 that may be used as a surface antigen for adult stem cells immunopurification.

Also, we show that stimulation of mesenchymal stem cells with CCL5 leads to stimulation of proliferation as assessed by the BrdU incorporation. This effect of CCL5 can be used to stimulate propagation of mesenchymal stem cells in vitro or stimulate mesenchymal stem cells in the process of isolation prior to grafting them to the recipient.

Finally, we show that stimulation of MSCs with CCL5 leads to elevation of genes that are characteristic for differentiation of cells into different lineages. We show that pretreatment of mesenchymal stem cells with CCL5 stimulates expression of marker genes for adipogenesis (PPARγ2, ADIPOQ, CFD and FABP4) and chondrogenesis (SOX9, COL2A1, NKX3.1 and MMP13) Since these genes are key regulators of adipocyte and chondrocyte differentiation, treatment of MSCs with CCR5 may have beneficial effects for differentiation of these and other lineages. Stimulation of chondrogenesis may have beneficial effects in particular for generation of articular chondrocytes in vitro and in vivo.

### Example 1

### Expression of CCL5 and its Receptors CCR1, CCR2, CCR3 and CCR5 in Human Adult Mesenchymal Stem Cells (AdMSCs) Using RT-PCR Analysis

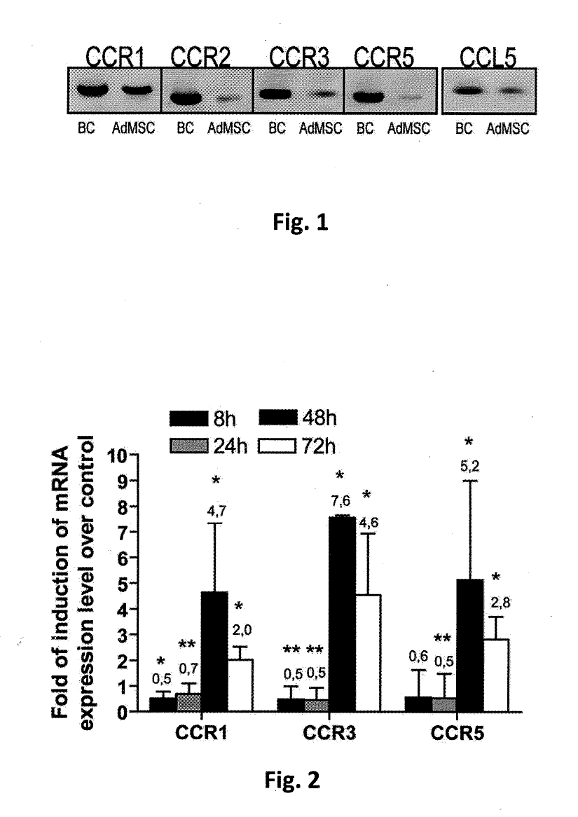

To confirm expression of CCR1, CCR2, CCR3, CCR5 and CCL5 in adult mesenchymal stem cells (MSCs) we analyzed the expression of CCL5 and its receptors CCR1, CCR2, CCR3 and CCR5 in AdMSCs and blood cells by RT-PCR using two independent pairs of primers. The data of a representative experiment are depicted in FIG. 1. Our results showed that CCL5, CCR1, CCR2, CCR3 and CCR5 are expressed in AdMSCs. Our RT-PCR data was confirmed using flow cytometry and immunofluorescence staining of AdMSCs cells.

We then analyzed the changes of the CCR1, CCR3 and CCR5 expression in response to CCL5 treatment. The AdMSCs were stimulated with CCL5 over 8, 24, 48 and 72 h. The levels of CCR1, CCR3 and CCR5 mRNA were measured in triplicates using qRT-PCR and normalized by GAPDH mRNA expression level. The average mean of three independent experiments±SEM is shown in FIG. 2. Our analysis revealed that the levels of CCR1, CCR3 and CCR5 expression increase after 48 h of CCL5 treatment. Our results were confirmed using Western blot analysis, which showed stimulation of expression of CCL5 receptors on the surface of AdMSCs following treatment with CCL5. Increased levels may be caused by activating transcription of the CCR1, CCR3 and CCR5 or increased proliferation of CCR1, CCR3 and CCR5 positive cells.

### Example 2

### Treatment of AdMSCs with CCL5 Stimulates Expression of IL-6, MMP1, MMP9 and Anti-Apoptotic Gene SURVIVINE

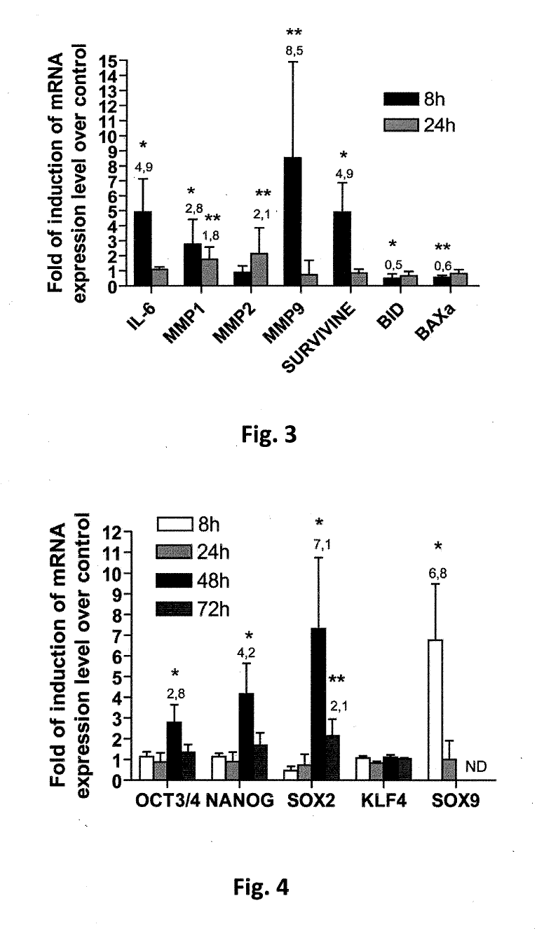

We also estimated the changes of several NFkB-dependent genes expression in AdMSCs in response to CCL5 stimulation. The cells were either stimulated or not with CCL5 over the indicated periods of time. The average means±SEM of the induction of genes expression levels over control obtained from independent experiments performed with three different pools of AdMSCs are presented in FIG. 3. We have found that expression levels of IL-6, MMP1, MMP9 and anti-apoptotic gene SURVIVINE were increased within 8 h of induction. The level of TIMP1 and TIMP2 was not changed. Besides, the expression of pro-apoptotic BID and BAXa genes was down-regulated. After 24 h of induction, the expression levels of all tested genes except MMP2 were decreased approximately to the control level. The level of MMP2 was up-regulated within 24 h of induction. Taking together, our data suggest that AdMSCs are able to re-modulate the extracellular matrix in response to CCL5 that in turn point to the possible activation of migratory mechanisms in AdMSCs upon CCL5 stimulation. Besides, CCL5 may be a positive regulator of AdMSC proliferation preventing their apoptosis.

### Example 3

CCL5 Stimulates Expression of Multipotency Related Genes in AdMSCs

We analyzed the expression levels of the multipotency genes OCT4, KLF4, SOX2, NANOG and SOX9 following treatment of AdMSCs with CCL5. We induced the AdMSCs with CCL5 during indicated periods of time and measured OCT4, KLF4, SOX2, NANOG and SOX9 mRNA expression levels in the induced and non-induced cells. The average data of three independent experiments performed with different pools of AdMSCs are depicted in FIG. 4. Our analyses revealed that the expression of multipotency genes OCT4, NANOG and SOX2 increased after 48 h of induction with CCL5. These data correlate with the increased level of CCR1, CCR3 and CCR5 expression during the same period of induction and again point to the possible association of multipotency and genes for CCL5 receptors.

### Example 4

CCL5 Stimulates Proliferation of AdMSCs

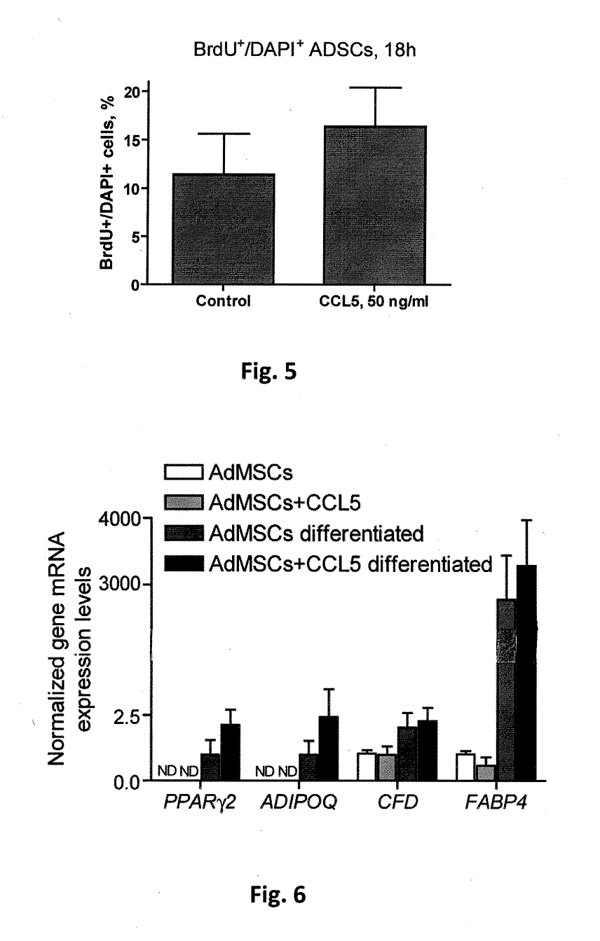

We analyzed the effect of CCL5 on proliferation of AdMSCs by treatment of AdMSCs with 50 ng/ml of CCL5 and 10 μM BrdU for 18 h, and assessed for a portion of BrdU+ cells by IF. AdMSCs exposed only to BrdU were used as a control. BrdU incorporation was assessed in at least 200 DAPI+AdMSCs isolated from three donors, and the data of cell counting are presented as an average mean of BrdU+/DAPI+ cells±SD in FIG. 5. Cell counting revealed that approximately 11.4±4.2% of AdMSCSs were positive for BrdU. Treatment with CCL5 led to increase of BrdU+AdMSCs to 16.4±4%. The portion of CCR1+ cells in AdMSCs treated with CCL5 for 48 h was assessed using flow cytometry. Amount of CCR1+ cells was increased approximately 28±14% in response to CCL5 signaling.

Immunofluorescence (IF). AdMSCs were plated on slides (BD Biosciences) at density of approximately 3000 cells/cm² and treated with 50 ng/ml of CCL5 and 10 μM BrdU for 18 h. Cells were washed 3 times with TBS and fixed in 4% paraformaldehyde in TBS for 15 min at RT and 15 min at +4° C. The cells were permeabilized with 0.3% Tween20 in blocking solution for 30 min and treated with 2N HCl for 8 min at RT with subsequent washing with TBS containing 0.5% BSA. The cells were incubated in blocking solution at +4° C. overnight. Cells were exposed to CCR1 antibody CKR-1 (C-20), and BrdU antibody mAbG3G4 (Developmental Studies Hybridoma Bank, University of Iowa, IA, USA) at dilutions 1:300 and 1:200, respectively, for 2 h at RT. The cells were washed 3 times for 10 min with TBS containing 0.5% BSA and incubated with Alexa Fluor 488 or Alexa Fluor 568 secondary antibodies (Molecular Probes, A11017 or A11079) (dilutions 1:1000) for 1 h at RT. CCR1 and secondary antibodies were diluted in blocking solution. BrdU antibody was diluted in blocking solution supplemented with 0.3% Triton X-100 (Sigma). Cells were mounted with Prolong Gold antifade reagent containing 4′,6-diamidino-2-phenylindole (DAPI) (Invitrogen). The cells were visualized under a fluorescence microscope Olympus BX61 with UPLan SApo 40× or 20× objectives.

Flow cytometry. Approximately 10⁵ cells were harvested by trypsinization, washed with PBS and blocked in 2% bovine serum albumin (BSA) (PAA Laboratories GmbH, Pasching, Austria) in PBS. Antibody against CCR1 (CKR-1 (C-20), Santa Cruz Biotechnology, sc-6125) was diluted 1:100 in the blocking solution. The cells were incubated with primary antibody for 1 h on ice, washed for 10 min with PBS containing 0.5% BSA and incubated with secondary antibody Alexa Fluor 488 or (A11017, Molecular Probes, Eugene Oreg., USA) diluted in PBS+4% BSA at 1:400 for 45 min on ice. AdMSCs stained only with secondary antibody were used as a nonreactive control. Cells were analyzed on Accuri™ C6 flow cytometer (BD Biosciences). Data acquisition and analysis were performed using BD Accuri C6 software (BD Biosciences).

### Example 5

### Pre-Treatment with CCL5 Enhances Subsequent Differentiation of AdMSCs

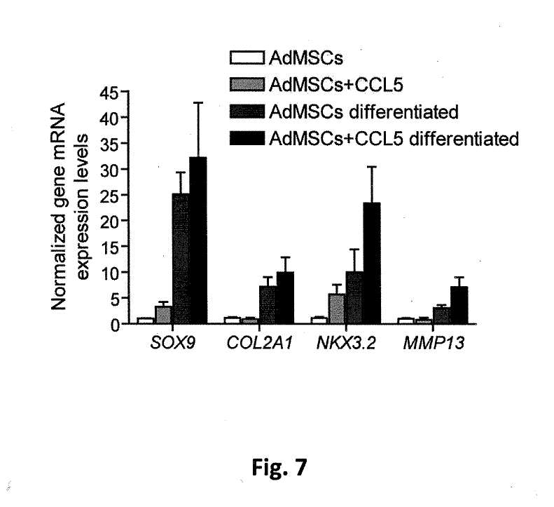

Stimulation of proliferation of CCR1+AdMSCs by CCL5 accompanied with increased OCT4, NANOG and SOX2 gene expression levels suggested that treatment of heterogeneous populations of AdMSCs with CCL5 leads to increase of a portion of multipotent or “real” stem cells in these populations. One of the criteria of stem cells is their differentiation ability. Adipogenic and chondrogenic differentiation potential of native or pre-treated with CCL5 AdMSCs was assessed using qRT-PCR. Expression levels of adipogenic markers PPARγ2, ADIPOQ, CFD and FABP4 and chondrogenic markers SOX9, COL2A1, NKX3.1 and MMP13 are shown in FIGS. 6 and 7, respectively. Expression of PPARγ2 and ADIPOQ was not detected in undifferentiated AdMSCs, therefore the level of these gene expression in differentiated non-treated with CCL5 AdMSCs was set as 1. Expression of other genes in non-differentiated and non-treated with CCL5 AdMSCs was set as 1. Data from other samples were calculated relative to 1. Pre-treatment of AdMSCs with CCL5 leads to increased adipogenic and chondrogenic differentiation capacity of AdMSCs.

Treatment of MSCs with CCL5. Prior induction of differentiation, AdMSCs were subjected to FBS starvation for 12 h and treated with 50 ng/ml of CCL5 in Light growth media (DMEM-LG containing 3% HI-FBS and 1% PEST) for 48 h, if indicated. Alternatively, Light growth media without CCL5 was used.

Differentiation. Adipogenic differentiation of AdMSCs was performed in normal growth media containing 10 μg/ml of human insulin, 1 μM dexamethasone, 0.5 mM IBMX and 10 μM indomethacin for 5 days. Chondrogenic differentiation of AdMSCs was conducted in DMEM-High glucose containing 10 ng/ml of TGFβ1 (Peprotech), 1×ITS supplement, 100 μM ascorbate-2-phosphate and 1 μM dexamethasone for 9 days. All chemicals were purchased form Sigma-Aldrich. The differentiation media were replenished every 3 days. The cells were collected by trypsinization and split for total RNA and protein isolation.

Quantitative RT-PCR. Total RNA was isolated using RNeasy micro kit (Qiagen, Valencia Calif., USA) according to the manufacturer's instructions. Complementary DNA was synthesized from 1 μg of total RNA using SuperScript™ III Reverse Transcriptase (Invitrogen) according to the manufacturer's protocol using Oligo(dT)20. We used 0.5 μl of cDNA per one PCR.

Quantitative RT-PCR (qRT-PCR) was performed using Platinum® SYBR® Green qRT-PCR SuperMix-UDG (Invitrogen). The levels of target gene mRNAs and mRNA of Glyceraldehyde 3-phosphate dehydrogenase (GAPDH) used for normalization were detected in triplicates with LightCycler® 480 Real-Time PCR System (Roche Applied Science, Basel, Switzerland). Data were analyzed using Lightcycler 480 software (Roche) and calculations were carried out using comparative CT method. Primers used in the current study are described in Ref. Kauts et al 2013 or listed in TABLE 1:

TABLE 1
PCR Primer Pairs

| | | |
|---|---|---|
| FABP4 s | (SEQ ID NO: 1) | AACCTTAGATGGGGGTGTCC |
| FABP4 as | (SEQ ID NO: 2) | TGGTTGATTTTCCATCCCAT |
| ADIPOQ s | (SEQ ID NO: 3) | TGCACAGGTTGGATGGCGGG |
| ADIPOQ as | (SEQ ID NO: 4) | GGGACCTTCAGCCCCGGGTA |
| CFD s | (SEQ ID NO: 5) | CACCGAGCGCTTGATGTGCG |
| CFD as | (SEQ ID NO: 6) | TCCCGGGCTTCTTGCGGTTG |
| PPARγ2 s | (SEQ ID NO: 7) | TCCATGCTGTTATGGGTGAA |
| PPARγ2 as | (SEQ ID NO: 8) | TCAAAGGAGTGGGAGTGGTC |
| COL2A1 s | (SEQ ID NO: 9) | GGAGAAACCATCAATGGTGGC |
| COL2A1 as | (SEQ ID NO: 10) | CTGCTTCGTCCAGATAGGCAATG |
| SOX9 s | (SEQ ID NO: 11) | CAGACGCACATCTCCCCCAAC |
| SOX9 as | (SEQ ID NO: 12) | CTCTCGCTTCAGGTCAGCCTTG |
| ACAN s | (SEQ ID NO: 13) | CACTGGCGAGCACTGTAACAT |
| ACAN as | (SEQ ID NO: 14) | TCCACTGGTAGTCTTGGGCAT |
| NKX3.2 s | (SEQ ID NO: 15) | CCGCTTCCAAAGACCTAGAGGA |
| NKX3.2 as | (SEQ ID NO: 16) | ACCGTCGTCCTCGGTCCTTGG |
| MMP13 s | (SEQ ID NO: 17) | CAGGAAACCAGGTCTGGAGAT |
| MMP13 as | (SEQ ID NO: 18) | CAAACTGTATGGGTCCGTTG |
| BMP4 s | (SEQ ID NO: 19) | AGGAAGAGCAGATCCACAGC |
| BMP4 as | (SEQ ID NO: 20) | TCGTGTCCAGTAGTCGTGTGA |
| CXCL12 s | (SEQ ID NO: 21) | CGTCAGCCTGAGCTACAGATGC |
| CXCL12 as | (SEQ ID NO: 22) | GTCTGTTGTTGTTCTTCAGCCG |

### Example 6

### Migration Capacity of AdMSCs is Associated with their Higher Differentiation Potential

The modified Boyden chamber test was used to assess the ability of AdMSCs to migrate in the presence of CCL5 and FBS. Analysis showed that ADMSCSs were able to migrate (data not shown). Migrated and non-migrated AdMSCs were isolated, expanded, and analyzed for OCT4, NANOG, BMP4, CXCR4, CXCL12, CCR1 and CCL5 mRNA expression using qRT-PCR. The normalized with GAPDH gene expression levels in non-migrated AdMSCs were taken as 1. Data from migrated AdMSCs were calculated relative to that (data not shown). Migrated AdMSCs express higher levels of OCT4, NANOG, BMP4, CXCR4, CXCL12, CCR1 and CCL5 mRNAs than their non-migrated counterparts. Adipogenic and chondrogenic differentiation capacity of in vitro expanded control or total initial population, non-migrated and migrated AdMSCs was assessed using WB and qRT-PCR (data not shown, for adipogenic and chondrogenic differentiation, respectively). The normalized with GAPDH levels of lineage-specific gene expression in differentiated control sample were set as 1. Data from non-migrated and migrated samples were calculated relatively to the control sample. Migrated and subsequently expanded AdMSCs demonstrated higher chondro- and adipogenic differentiation capacity compared with total initial population and non-migrated cells. Taken together these data suggest that migratory capacity is attributing of AdMSCs possessing higher stemness-related properties.

Cell migration assay. Cell migration assay was performed using NUNC™ polycarbonate Cell Culture Inserts with 8 μm pore size. AdMSCs were subjected to overnight serum-starvation in DMEM-LG with subsequent trypsinization and seeding of approximately 180,000 cells per insert in DMEM-LG. DMEM-LG supplemented with 2% of FBS and 50 ng/ml of CCL5 was added to the lower chamber. Migration assay was performed at 37° C. and 5% CO₂ for 4 h. Base medium without any supplement was used as a negative control. Migratory cells on the membranes were stained and visualized using a Zeiss Axiovert microscope with 20× objective.

Isolation of migrated and non-migrated cells. Non-migrated cells were collected from the upper side of a membrane using scraper and transferred to the cell culture plate containing normal cell growth medium. The upper side of a membrane was additionally purified by cotton swab. The membrane was cut out from the insert and placed into the Falcon tube containing trypsin-EDTA. Migrated cells were isolated by trypsinization for 5 min at RT with subsequent neutralization with Normal growth medium and centrifugation for 5 min at 1200 rpm. The membrane was removed from the tube, and the cells were resuspended in normal growth medium and transferred to the cell culture plate. The native population of AdMSCs exposed to migratory medium for 4 h and afterwards grown in normal growth media was used as a control.

What is claimed is:

1. A method of increasing expression of a chemokine receptor in a population of mesenchymal stem cells comprising:
a) providing an in vitro population of human mesenchymal stem cells; and
b) administering CCL5 to the population in an amount sufficient to increase expression of a chemokine receptor selected from the group consisting of CCR1, CCR2 and CCR5 in the population.

2. The method according to claim 1, wherein the method increases expression of CCR1, CCR3 and CCR5 in the population of cells.

3. The method according to claim 1, further comprising culturing the population of cells for about 48 hours after the step of administering CCL5 to achieve a higher level of expression of the chemokine receptor compared to a same population of cells cultured for about 24 hours and for about 72 hours after the step of administering CCL5.

4. A method of increasing expression of a NFkB-dependent gene selected from the group consisting of IL-6, MMP1, and MMP9 in a population of mesenchymal stem cells, the method comprising:
a) providing a in vitro population of human mesenchymal stem-like cells; and
b) administering CCL5 to the population in an amount sufficient to increase expression of a NFkB-dependent gene selected from the group consisting of IL-6, MMP1, and MMP9.

5. The method according to claim 4, wherein expression of an anti-apoptotic gene SURVIVINE is increased.

6. The method according to claim 5, the method further comprising culturing the population of cells for about 8 hours after administering CCL5 to obtain a higher level of expression of IL-6, MMP1, MMP9 and SURVIVINE and culturing the population of cells for about 24 hours to obtain a higher level of expression of MMP2.

7. A method of inducing expression of multipotency related genes selected from the group consisting of OCT3/4, NANOG, SOX 2, KLF4, AND SOX9 in mesenchymal stem cells, the method comprising:
a) providing a in vitro population of human mesenchymal stem cells; and
b) administering CCL5 to the population in an amount sufficient to induce expression of one or more multipotency related genes selected from the group consisting of OCT3/4, NANOG, SOX 2, KLF4, and SOX9.

8. The method according to claim 7, further comprising the step of culturing the population for about 48 hours from the step of administering CCL5 to achieve a higher level of expression of OCT 3/4, NANOG, and SOX2 compared to about 8 hours, 24 hours and 72 hours after the step of administering CCL5.

9. A method of stimulating proliferation of mesenchymal stem cells comprising:
a) providing an in vitro population of human mesenchymal stem cells; and
b) administering CCL5 to the population in an amount sufficient to stimulate proliferation.

10. A method stimulating differentiation of mesenchymal stem cells, comprising:
a) providing an in vitro population of human mesenchymal stem cells; and
b) administering CCL5 to the population in an amount sufficient to stimulate differentiation of mesenchymal stem cells.

11. The method according to claim 10, wherein the population of cells is cultured for 12 hours with CCL5 followed by differentiation into adipogenic or chondrogenic lineages.

12. The method according to claim 11, further comprising determining expression levels of PPARγ2, ADIPOQ, CFD and FABP4 to confirm an adipogenic and lineage.

13. The method according to claim 11, further comprising determining expression levels of SOX9, COL2A1, NKX3.1 and MMP13 to confirm a chondrogenic lineage.


SEQUENCE LISTING (22 sequences)

SEQ ID NO: 1 - DNA, 20 bp, artificial sequence
  synthetic construct
aaccttagatgggggtgtcc

SEQ ID NO: 2 - DNA, 20 bp, artificial sequence
  synthetic construct
tggttgattttccatcccat

SEQ ID NO: 3 - DNA, 20 bp, artificial sequence
  synthetic construct
tgcacaggttggatggcggg

SEQ ID NO: 4 - DNA, 20 bp, artificial sequence
  synthetic construct
gggaccttcagccccgggta

SEQ ID NO: 5 - DNA, 20 bp, artificial sequence
  synthetic construct
caccgagcgcttgatgtgcg

SEQ ID NO: 6 - DNA, 20 bp, artificial sequence
  synthetic construct
tcccgggcttcttgcggttg

SEQ ID NO: 7 - DNA, 20 bp, artificial sequence
  synthetic construct
tccatgctgttatgggtgaa

SEQ ID NO: 8 - DNA, 20 bp, artificial sequence
  synthetic construct
tcaaaggagtgggagtggtc

SEQ ID NO: 9 - DNA, 21 bp, artificial sequence
  synthetic construct
ggagaaaccatcaatggtggc

SEQ ID NO: 10 - DNA, 23 bp, artificial sequence
  synthetic construct
ctgcttcgtccagataggcaatg

SEQ ID NO: 11 - DNA, 21 bp, artificial sequence
  synthetic construct
cagacgcacatctcccccaac

SEQ ID NO: 12 - DNA, 22 bp, artificial sequence
  synthetic construct
ctctcgcttcaggtcagccttg

SEQ ID NO: 13 - DNA, 21 bp, artificial sequence
  synthetic construct
cactggcgagcactgtaacat

SEQ ID NO: 14 - DNA, 21 bp, artificial sequence
  synthetic construct
tccactggtagtcttgggcat

SEQ ID NO: 15 - DNA, 22 bp, artificial sequence
  synthetic construct
ccgcttccaaagacctagagga

SEQ ID NO: 16 - DNA, 21 bp, artificial sequence
  synthetic construct
accgtcgtcctcggtccttgg

SEQ ID NO: 17 - DNA, 21 bp, artificial sequence
  synthetic construct
caggaaaccaggtctggagat

SEQ ID NO: 18 - DNA, 20 bp, artificial sequence
  synthetic construct
caaactgtatgggtccgttg

SEQ ID NO: 19 - DNA, 20 bp, artificial sequence
  synthetic construct
aggaagagcagatccacagc

SEQ ID NO: 20 - DNA, 21 bp, artificial sequence
  synthetic construct
tcgtgtccagtagtcgtgtga

SEQ ID NO: 21 - DNA, 22 bp, artificial sequence
  synthetic construct
cgtcagcctgagctacagatgc

SEQ ID NO: 22 - DNA, 22 bp, artificial sequence
  synthetic construct
gtctgttgttgttcttcagccg